(19) United States
(12) Patent Application Publication
(10) Pub. No.: US 2022/0400251 A1
(43) Pub. Date: Dec. 15, 2022

(54) CONTEXT ENABLED MACHINE LEARNING

(71) Applicant: DYNAM.AI, Inc., San Diego, CA (US)

(72) Inventor: Dimitry FISHER, San Diego, CA (US)

(21) Appl. No.: 17/839,904

(22) Filed: Jun. 14, 2022

Related U.S. Application Data
(60) Provisional application No. 63/210,871, filed on Jun. 15, 2021.

(51) Int. Cl. H04N 19/105; G06N 20/00; G06K 9/00

(52) U.S. Cl. CPC H04N 19/105; G06N 20/00; G06K 9/00536

(57) ABSTRACT

Certain aspects of the present disclosure provide techniques for generating context-aware inferences using a machine learning model. The method generally includes receiving a time-series data sequence and a contextual model specifying characteristics of how objects behave in an environment in which the time-series data sequence was captured. A feature data set from the contextual model is extracted using a first machine learning model. Generally, the extracted feature data set comprises a representation of the specified characteristics of how objects behave in the environment. A future state of an object in the environment is predicted using the time-series data sequence and the extracted feature data set representing the specified characteristics of how objects behave in the environment as input into a second machine learning model. One or more actions are taken based on the predicted future state of the object in the environment.

### CROSS-REFERENCE TO RELATED APPLICATIONS

This Application claims the benefit of and priority to U.S. Provisional Patent Application Ser. No. 63/210,871, entitled “Physics wrapper apparatus and methods,” filed Jun. 15, 2021, the contents of which are hereby incorporated by reference in their entirety.

### INTRODUCTION

Aspects of the present disclosure relate to machine learning models, and more specifically, to machine learning models that generate inferences based on input data and contextual information associated with the input data.

### BACKGROUND

Machine learning models used in artificial intelligence systems generally allow machines to learn to solve a problem from a training data set including data from an environment in which the machine learning model is to be used. These machine learning models may be trained using supervised learning techniques in which each sample in the training data set is labeled with one of a plurality of classifications assigned to the sample (which may be performed manually or automatically), using unsupervised learning techniques in which the training data set is unlabeled and the machine learning model learns to recognize similarities between samples in the training data set, or semi-supervised learning techniques in which both labeled and unlabeled data is used to train the machine learning model. However, in each of these situations, the machine learning models may be limited by the data sets used to train these machine learning models.

Because machine learning models may be limited by the data sets used to train these machine learning models, these machine learning models may be useful in generating inferences only for data that is similar to that in the training data set used to train these models. However, these machine learning models may have poor performance on data that is different from the data used to train these machine learning models, such as so-called out of distribution (00D) data. Further, these machine learning models may be unaware of other information that may influence how objects, whether in a physical or a virtual environment, act in relation to other objects. To the extent that machine learning models are trained to predict a future state of an object, such training may also be limited by the scenarios included in the training data set and may not be generalizable to other unseen scenarios.

According, techniques are needed to improve the accuracy of inferences generated by machine learning models.

### BRIEF SUMMARY

Certain embodiments provide a computer-implemented method for generating inferences using a machine learning model. The method generally includes receiving a time-series data sequence and a contextual model specifying characteristics of how objects behave in an environment in which the time-series data sequence was captured. A feature data set from the contextual model is extracted using a first machine learning model. Generally, the extracted feature data set comprises a representation of the specified characteristics of how objects behave in the environment. A future state of an object in the environment is predicted using the time-series data sequence and the extracted feature data set representing the specified characteristics of how objects behave in the environment as input into a second machine learning model. One or more actions are taken based on the predicted future state of the object in the environment.

Certain embodiments provide a computer-implemented method for training a machine learning model. The method generally includes receiving a training data set including a plurality of time-series data sequences. A first machine learning model is trained to extract a feature data set representing characteristics of how objects behave in an environment in which the training data set was captured based on a contextual model specifying the characteristics of how objects behave in the environment. A second machine learning model is trained to predict a future state of an object in the environment based on the training data set and the feature data set representing the characteristics of how objects behave in the environment.

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates an example system in which machine learning models are trained and used to generate inferences based on contextual information associated with an environment in which the inferences are generated, according to aspects of the present disclosure.

FIG. 2 illustrates an example pipeline for predicting a future state of a scene, represented by one or more images, based on motion data and visual data input into a feature extractor/encoder and a prediction module, according to aspects of the present disclosure.

FIGS. 3A through 3C illustrate example pipelines for predicting a future state of objects in an environment based on extracting contextual information about how objects behave in an environment from an input signal, according to aspects of the present disclosure.

FIG. 4 illustrates an example environment in which a physics (or contextual) model is executed within a context wrapper, according to aspects of the present disclosure.

FIGS. 5A and 5B illustrate example environments in which a context-aware machine learning model is deployed, according to aspects of the present disclosure.

FIGS. 6A and 6B illustrate examples environments in which context-aware machine learning models are trained and used in inference, according to aspects of the present disclosure.

FIG. 7 illustrates example operations for generating context-aware inferences using a machine learning model, according to aspects of the present disclosure.

FIG. 8 illustrates example operations for training a machine learning model to generate context-aware inferences, according to aspects of the present disclosure.

FIG. 9 illustrates an example system on which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Machine learning models are powerful tools that allow computing systems to learn relationships between different inputs from a set of training data and infer a future state of an environment and/or objects within an environment. However, machine learning models may be limited to making accurate inferences that are similar to the training data used to train these models and may be sensitive to changes in data that, to a human, would not change the result of an inference. For example, given an input image x, a machine learning model may classify an object in the image x accurately (e.g., may infer that the object in the image xis of the correct class of objects). However, if some random noise, e.g., represented by the equation sign(∇ₓJ(θ, x, y) is added to image x, the resulting classification generated by the machine learning model may be different from the accurate classification, even though the combined image and noise may be perceptually unchanged, or minimally changed, from the original image.

Further, machine learning models may be unable to infer information about a future state of an environment because of limitations of the training data sets used to train these machine learning models. In machine learning models that are trained using examples of various situations defined in terms of time-series data, the machine learning models may not be able to generalize from the sequences of actions in the training data set to a more generalized set of rules that define how objects act and interact within an environment. For example, a machine learning model may learn that in a specific situation, an object will fall down over time, but the machine learning model may not learn why the object fell down. The machine learning model may thus predict that the object will fall down in this specific situation (e.g., when the input time-series data set is similar to a time-series data set used to train the machine learning model), but may not be able to predict whether an object will fall down in other situations defined by different data sets, as the machine learning model may not be able to extrapolate sufficient information to determine that similar rules apply to different scenarios.

To attempt to improve the accuracy of machine learning models, natural language understanding and knowledge databases, statistical information (regularities or irregularities) derived from a training data set, and data augmentation may be used. However, in each of these examples, information about an environment may be hard-coded into the machine learning model, explicitly defined in a training data set (e.g., in machine learning models trained using transfer learning techniques in which additional data is manually selected for inclusion based on a similarity of the additional data to the original data and task for which a model is trained), or synthetically generated from the original data in the training data set. Thus, these machine learning models may still be limited to generating accurate inferences in situations in which the input data has previously been seen in a training data set or is sufficiently close to data in the training data set.

Aspects of the present disclosure provide techniques for training and using machine learning models to generate inferences based on an input data set and contextual information about the environment in which the inferences are made. Generally, the contextual information may be information extracted from a contextual model (also referred to as a “physics model” in some examples) that specifies the characteristics of how objects behave in an environment, which may be a physical environment or a virtual environment. These characteristics may include, for example, information defining how objects move in relation to other objects in in an environment, information defining how objects are transformed or destroyed by other objects in the environment, information about forces acting on objects within an environment (e.g., a Newtonian physics model) or other information defining interactions between different objects within the environment. By introducing contextual information about the environment in which an inference is to be made as an input into a machine learning model, inference accuracy may be improved relative to typical machine learning models and out of distribution-related defects in model performance may be mitigated. Based on the contextual information about the environment in which the inferences are made, the machine learning models discussed herein may make inferences (e.g., predict a future state of the environment) that take into account generalized information about how objects interact with each other within the environment such that the inferences are consistent with how objects actually behave within the environment. For example, in the image noise example discussed above, the contextual information may allow for image noise to be disregarded so as to allow for inference over the entirety of the image instead of inference based on individual portions of the image which may be substantially affected by the introduction of noise. In the Newtonian physics example discussed above, the contextual information may allow for the machine learning models to take into account the effects of gravity on unsupported objects in the environment so that the trajectory of these objects over time may be accurately predicted (as opposed to being predicted in a zero-gravity environment in which gravitational forces do not exist and are not imparted to the objects in the environment). Thus, aspects of the present disclosure may improve the accuracy of inferences generated by machine learning models.

Further, aspects of the present disclosure may efficiently perform anomaly detection within the environment based on a difference between the actual future state of the environment and a predicted future state of the environment. As discussed, inferences generated by these machine learning models may be consistent with how objects actually behave within the environment. Because these inferences may be consistent with how objects behave in the environment, a divergence between an inferred (or predicted) future state of objects in the environment and the actual future state of objects in the environment may indicate that an anomaly exists within the environment, and comparisons between the predicted future state of the objects and actual future state of the objects may be performed with minimal additional computational expense. Thus, aspects of the present disclosure may also improve the efficiency of anomaly detection using machine learning models.

Example Context-Aware Inference in Machine Learning Models

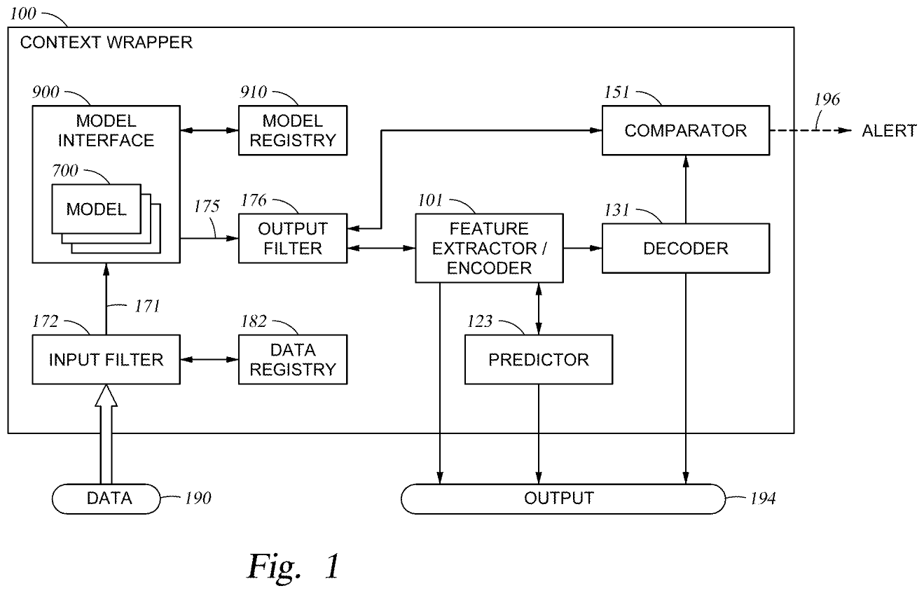

FIG. 1 illustrates an example context wrapper 100 in which machine learning models are trained and used to generate inferences based on contextual information associated with an environment in which the inferences are generated. As illustrated, context wrapper 100 includes a feature extractor/encoder 101, predictor 123, decoder 131, comparator 151, data input pipeline 171, data input filter 172, model data output pipeline 175, model data output filter 176, data registry 182, a plurality of physics models 700, physics model interface 900, model registry 910, and a plurality of input/output interfaces over which various data can be input into and output from context wrapper 100.

Generally, model interface 900 receives input data 190, which may be filtered through input filter 172, through data input pipeline 171. Input filter 172 may filter the input data 190, for example, to remove data that may not be relevant to a given model, to remove duplicative data, to sample the data provided to model interface 900, and the like. Model interface 900 passes the input to one or more of the plurality of physics models 700 for processing. In some aspects, model interface 900 may implement additional functionality for modifying a data set, adjusting one or more models 700 (e.g., adjusting parameters of these models), load balancing, and other operations that may be performed to manage operations within context wrapper 100.

Generally, to initiate operations through context wrapper 100, data 190 and a specification of the one or more models 700 (also referred to as “physics models” or “contextual models”) to be used to provide contextual information about the environment in which a machine learning model is to predict a future state may be provided directly to context wrapper 100 or selected from data registry 182 and/or model registry 910 based on user input. The specification of the one or more models may be an identifier of a model 700 in model registry 910, mathematical equations defining the model 700, or other information defining the model 700. During operations, model results generated by the selected model(s) 700 may be passed to feature extractor/encoder 101, which may be trained to extract features of the model 700 and output the features to predictor 123, as illustrated. In some aspects, feature extractor/encoder 101 may be trained to extract the features defining the context of the environment in which predictor 123 is to make a prediction based on the model results generated from input data 190 (which may be actual data or a simulation)

Feature extractor/encoder 101 and predictor 123 may be structured as different machine learning models that may be jointly trained or separately trained. Generally, feature extractor/encoder 101 may be trained to extract a feature set representing characteristics of how objects behave in an environment. The feature set representing the characteristics of how objects behave in the environment may be extracted from a model 700 (also referred to as a “contextual model” or “physics model”) specifying the characteristics of how objects behave in the environment. The feature extractor/encoder 101 may be, for example, an encoder-decoder model trained to extract a feature data set as one or more points in a feature space and a decoder trained to construct an approximation of a scene from the feature data set. In some aspects, feature extractor/encoder may be trained to extract the feature set representing characteristics of how objects behave in the environment based on a model 700 that receives inputs from various devices, such as sensors, imaging devices, or the like, and constructs a simulation of the environment based on the received inputs and various a priori defined rules within model 700 that define how objects behave within the environment. Feature extractor/encoder 101 may thus be trained to generate the resulting feature set based on the input data and the rules that apply to objects within the environment. In a physical environment, these rules may include rules related to forces imparted on objects in the environment (e.g., gravitational forces, external forces, etc.), laws of motion (e.g., Newtonian laws of motion dictating how and when objects move), and the like. In a virtual environment, these rules may include programmatic rules that define how different virtual objects interact with each other and with the virtual environment. As used herein, a “physics model” or “contextual model” may describe laws of nature and laws of physics, and may also or alternatively be other models, such as economic models, behavioral models, chemical models, financial models, mathematical models, engineering models, or other models, analytic or computational, implemented in software and/or hardware. The resulting feature set may be, for example, a set of features and feature coefficients as a function of time that defines how objects act with each other and within the environment over time.

Meanwhile, predictor 123 may be an encoder-decoder model separate from the model(s) used to implement feature extractor/encoder 101. When structured as an encoder-decoder model, predictor 123 may generally include an encoder trained to encode an input of data 190 and a feature data set representing characteristics of a model 700 defining how objects behave in the environment, a predictor trained to predict a future state of a time-series data sequence based on the encoded input and the feature data set representing characteristics of how objects behave in the environment, and a decoder trained to construct an approximation of a scene from the predicted future state of the time-series data sequence. In some aspects, the second machine learning model may be trained to minimize a difference between the predicted future state of the time-series data sequence and the actual time-series data sequence in the training data set. For example, data 190 used to train predictor 123 may be a time-series data sequence including n samples, with sample 0 representing an initial sample in the time-series data sequence and sample n−1 representing the final sample in the time-series data sequence. To train predictor 123, m samples, where m<n, may be used as an input data set, and samples m through n−1 may be used to refine predictor 123. For example, predictor 123 may be trained to predict the state of environment at the times corresponding to samples m through n−1 (assuming indexing from 0 through n−1) based on the set of m samples. Predictions of the state of the environment at times associated with samples m through n−1 may be compared to the actual states of the environment for the times corresponding to samples m through n−1 (e.g., to “ground truth” data included in data 190), and predictor 123 may be refined to minimize the difference between the predicted states of the environment and the actual states of the environment at the times corresponding to samples m through n−1.

In some aspects, the feature extractor/encoder 101 and predictor 123 may be trained using semi-supervised learning techniques. In semi-supervised learning, the feature extractor/encoder 101 and predictor 123 may be trained using time-series data sequences including n samples, with each sample corresponding to a state of an environment at a given time. The training data set may include a plurality of time-series data sequences, with each sequence including unlabeled data. The feature extractor/encoder 101 and predictor 123 may thus attempt to learn various properties of the time-series data sequences in the training data set based on the structure of each time-series data sequence. Data corresponding to the future state of the environment may be used as ground truth data against which feature extractor/encoder 101 and predictor 123 are trained to generate predictions of the future state of the environment. As discussed, the feature extractor/encoder 101 and predictor 123 may thus be trained to minimize a difference between the actual future state of the environment and the predicted future state of the environment.

Because there may be an indeterminate number of situations that may arise within any given environment, hard-coding rules may not be possible for every specific situation that may arise within the environment. Further, in any given environment, the future state of objects in the environment may be weakly constrained. That is, no one rule may define how objects act within the environment; rather, how objects act within the environment may be affected by or otherwise defined in relation to many factors or rules within the environment. For example, in a physical environment, objects are generally affected by the gravitational force (e.g., an environment rule in the physical environment that defines gravity as a downward-acting force with an acceleration of 9.8 meters per second squared), but also may be affected by other external forces that impart other forces on these objects and cause these objects to move. By training feature extractor/encoder 101 and predictor 123 to predict a future state of objects in an environment based on features extracted from a model, such as model 700 or other contextual or physics models, specifying how objects behave within an environment, the feature extractor/encoder 101 and predictor 123 may accurately predict the state of objects in the environment without using hard-coded rules and taking into account the many forces that may act on an object within the environment.

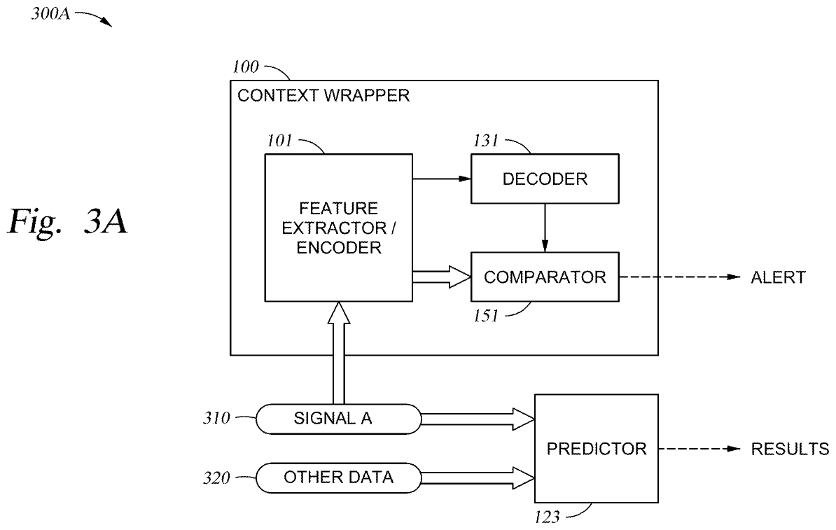

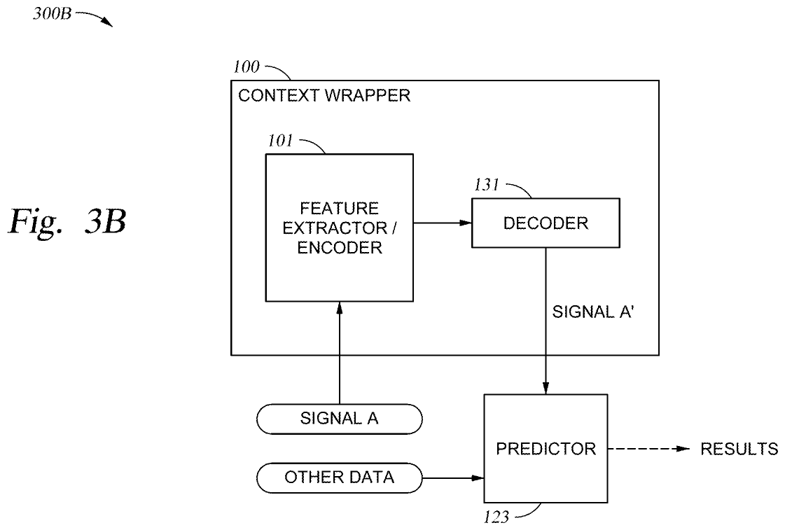

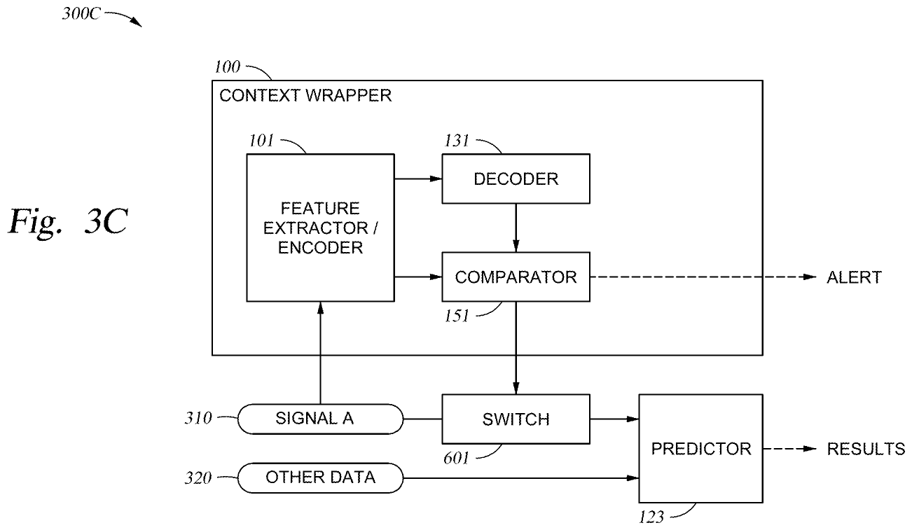

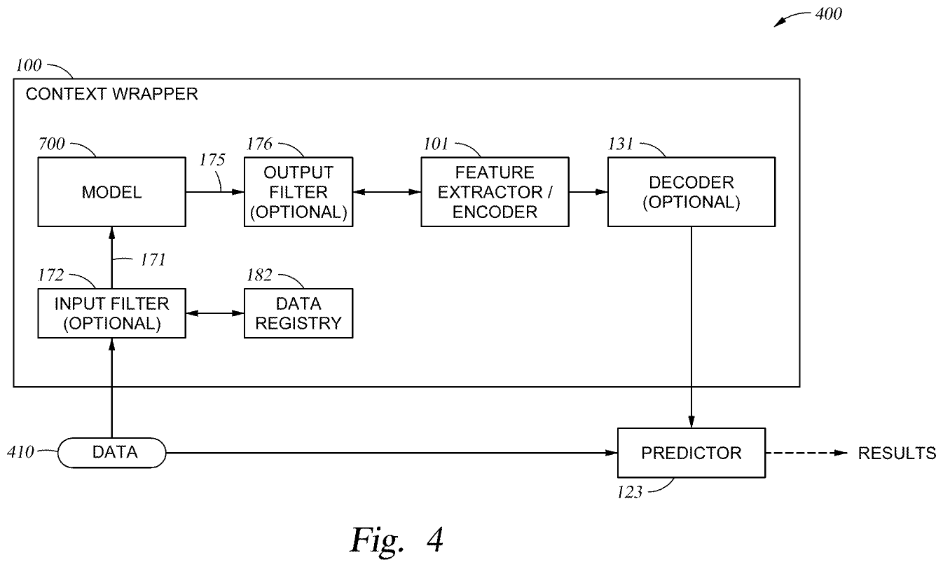

Feature extractor/encoder 101 and predictor 123, after being trained and deployed, predict a future state of objects in an environment, and thus, the future state of the environment, based on input data 190 (e.g., time-series data representing data captured over a period of time) and one or more of models 700 that specify the characteristics of how objects behave in the environment. The resulting predictions generated by feature extractor/encoder 101 and predictor 123 may be output for use in performing various actions with respect to one or more objects within the environment. In some aspects, predictions may be generated by feature extractor/encoder 101 and predictor 123 based on the input data 190 only. In such a scenario, the model 700 may be used in training and testing of the feature extractor/encoder 101 and predictor 123, but need not be used in to generate a prediction or deployed for use. In some aspects, feature extractor/encoder 101 and predictor 123 may use the output of the model 700 to generate a prediction without the use of input data 190. For example, feature extractor/encoder 101 and predictor 123 may produce a set of features or data that is then provided to predictor 123 external to context wrapper 100, as illustrated in FIGS. 3A, 3B, 3C, and 4 and discussed in further detail below. In some aspects, as discussed below, the feature extractor/encoder 101 and predictor 123 may be trained jointly with another predictor. In some aspects, the feature extractor/encoder 101 and predictor 123 may be pre-trained or trained before training of another predictor commences.

In some aspects, feature extractor/encoder 101 may be configured to output features and feature coefficients, among other outputs, as inputs into predictor 123. The features and feature coefficients may be defined as a function of time such an input into model 700 can be reconstructed based on the features, feature coefficients, and a time value associated with a time at which the input was generated (e.g., in relation to an arbitrarily defined starting time). The outputs generated by feature extractor/encoder 101 may be used by predictor 123, in conjunction with input data 190, to generate a prediction of a future state of the environment as an output of context wrapper 100. This prediction of the future state may be, for example, data that describes attributes of various objects within the environment at some future point in time based on an input of current and historical data defining previous interactions of these objects within the environment. In some aspects, a prediction made by predictor 123 may be a prediction in a latent space, which may have reduced dimensionality relative to a dimensionality of the inputs provided to models 700. In such a case, a decoder 131 may be used to decode the prediction from the latent space into the same (or similar) space as the inputs provided as inputs to models 700 through model interface 900. For example, a prediction made in a latent space may be a representation that may be compressed to reduce the amount of data based on which predictions are to be made; however, because the representation in the latent space may not be understandable outside of a space in which various machine learning models can understand, decoder 131 may transform the representation in the latent space to a human-understandable output that describes the predicted future state of the environment.

Feature extractor/encoder 101 and predictor 123 may be implemented in various environments and may generate inferences, or predictions of a future state of one or more objects, with respect to these environments. For example, feature extractor/encoder 101 and predictor 123 may be used in autonomous (self-driving) vehicles to identify objects to avoid and predict if and when objects will enter the path of travel of these autonomous vehicles, given a model 700 that defines how objects move within a physical environment (e.g., a physics model defining characteristics of human locomotion, of vehicle motion, etc.). The resulting predictions may be used to generate various control signals that control the steering, braking, and acceleration functions of the autonomous vehicles. For example, when predictor 123 generates a prediction that an object, such as a person, will enter the path of travel of an autonomous vehicle, control signals may be generated to cause the autonomous vehicle to stop, to steer in a manner that avoids contact with the object, and the like.

In another example, predictor 123 may be used in various medical diagnostics and treatment systems. For example, a medical diagnostics system may use predictor 123 to identify tumors or other anomalies in a series of medical scans of a patient, given a model 700 defining properties of tumor growth over time, and perform various predictions with respect to the identified tumors or other anomalies. A model may define how these tumors grow, given physical constraints, the supply of blood and nutrients to these tumors, interactions between tumors and healthy tissue, and the like. The resulting predictions generated by feature extractor/encoder 101 and predictor 123 may generate various imagery showing the predicted growth of a tumor and the predicted boundaries of the tumor over time and may be output for use in various diagnostics, detection, and treatment operations. For example, a series of predictions, played back in reverse, may be used for early detection of solid tumors. In this example, playing the series of predictions in reverse from an ending state may allow for identification of an area in which a tumor is likely to begin growing, as the series of predictions may show a realistic progression of tumor growth over time and a reverse playback of these predictions may allow for this progression sequence to be rewound to identify the location from which a tumor is likely to begin growing. In another example, the predictions made by predictor 123 can be used to improve the accuracy of diagnoses, as the predictor 123 may be trained to differentiate malignancies, such as solid tumors that grow predictably, versus other pathologies that may be benign or may be indicative of a non-cancerous condition. In yet another example, the series of predictions may be used to build three-dimensional models of a tumor and the surrounding areas, which may account for the position in which a patient is in, patient and surgical tool motion, edemas caused by these tumors, and the like. Based on these three-dimensional models, areas may be calculated the precisely target a tumor for treatment (e.g., via radiotherapy, surgical resection/excision, etc.).

Other examples of predictions that may be generated by predictor 123 within a physical environment may include various predictions used in controlling robotic devices, in performing non-destructive testing of various devices, in other computer vision tasks, such as object location or prediction within a spatial environment, or other tasks in which predictions may be made with respect to a physical environment.

It should be recognized that predictor 123 may also or alternatively be used to generate context-aware inferences of a future state of objects in virtual environments. When predictions are made of the future state of objects in a virtual environment, these predictions may be output to one or more display devices for visualization in augmented reality, extended reality, and/or virtual reality environments. In another example, these predictions may be used within video games or other interactive environments for various purposes, such as targeting, triggering actions within these interactive environments, or the like. The predicted future state of the object may include various properties of the object within the virtual environment, such as a three-dimensional location (e.g., a location along the x, y, and z axes), velocity, direction of movement, and the like. The predicted future state may be used to load, or pre-load, various multimedia assets that may be presented within the virtual environment at a later point in time, which may reduce latency involved in presenting these multimedia assets and allow for these multimedia assets to be loaded into memory when it is likely that such assets will be used, in turn reducing memory overhead for assets that may only be sparingly used within the virtual environment.

Because feature extractor/encoder 101 and predictor 123 may be trained to generate predictions of the future state of an environment that are based on contextual information that defines how objects interact with each other within an environment, comparator 151 can compare the actual and predicted future states of the environment to determine whether the environment in which feature extractor/encoder 101 and predictor 123 are operating is anomalous, relative to states of the environment which were included in training data sets used to train feature extractor/encoder 101 and predictor 123. To determine whether an anomaly exists within an environment, a prediction error may be calculated between an actual future state of the environment and the predicted future state of the environment. If the prediction error exceeds a threshold prediction error, comparator 151 may determine that an anomalous situation exists within the environment in which inferences are being generated, relative to situations which have been included in the data set used to train feature extractor/encoder 101 and predictor 123. Based on determining that an anomalous situation exists within the environment in which inferences are being generated, comparator 151 may indicate that the predictions generated by feature extractor/encoder 101 and predictor 123 may be inaccurate.

In some aspects, to calculate the prediction error between the actual future state of the environment and the predicted future state of the environment, comparator 151 may generate a prediction error heat map including a plurality of segments. Each segment may reflect a difference between a respective segment in an image representing the predicted future state and an image representing the actual future state. The resulting prediction error may be calculated as a score based on a value of each segment in the prediction error heat map. For example, the prediction error may be calculated as an average error over the segments in the prediction error heat map, as a cumulative error score, or using other techniques that quantify the difference between the actual future state of the environment and the predicted future state of the environment.

Comparator 151 may identify anomalies in various situations. For example, comparator 151 may be used to monitor the health and operational state of various machines. Given a series of monitor readings or test results for various physical components within these machines, comparator 151 can determine whether a predicted operational state of a machine and an actual operational state of a machine at a future point in time have diverged. If the predicted operational state of the machine and the actual operational state of the machine have diverged, various actions may be taken to recalibrate and repair the machine.

In another example, comparator 151 may be used in various medical applications to monitor disease progression. In monitoring disease progression, comparator 151 can use a prediction of a disease state based on diagnostic codes, procedure codes, and monitored data to identify divergences between predicted codes or monitored data and the actual codes or monitored data generated for a patient. These divergences may be used to determine whether the predicted progression of a disease has stalled or accelerated, and thus may be used to determine future treatment plans (e.g., to continue to impede disease progression, change treatment plans in response to an acceleration of disease progression, etc.).

In still another example, in a monitoring environment (e.g., during a surgical procedure), comparator 151 may be used to determine when the vital signs for a patient are diverging from a predicted set of vital signs and thus allow for early action to be taken to stabilize the patient. Comparator 151 may identify a divergence based on monitor readings of patient vital signs (e.g., electrocardiogram traces, pulse rate, blood pressure, etc.) that diverge from predicted readings of these vital signs, and may do so prior to such readings triggering one or more alarms. When a divergence is detected, comparator 151 can generate an alert that may trigger execution of various stabilization procedures for the patient, trigger a temporary pause in treatment, and the like.

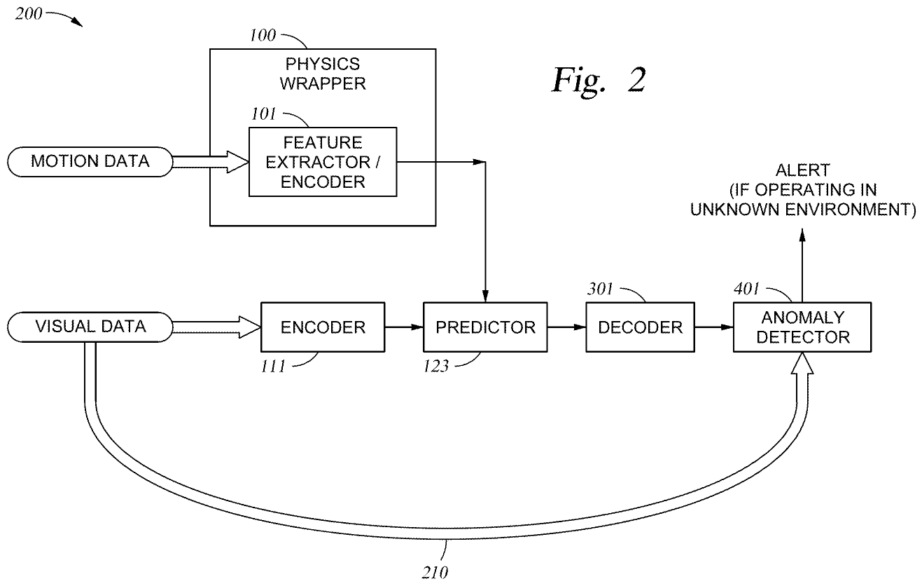

FIG. 2 illustrates an example 200 in which a future state of a scene, represented by one or more images, is predicted based on motion data and visual data input into a feature extractor/encoder 101 and a prediction module 123. As illustrated, motion data may be encoded by feature extractor/encoder 101 in context wrapper 100, and the encoded motion data may be provided as an input into a predictor 123. Generally, the encoded motion data may include a representation of the motion data over time and the properties of such motion data. Meanwhile, visual data, such as one or more images of a scene, may be encoded by an encoder 111 into a latent space representation of these images. Generally, the encoder 111 may be a machine learning model trained to generate a compact representation of each image input into encoder 111 that represents various features of various objects, such as a position of an object in a frame, velocity relative to the position of the object in a previous frame (which may be derived based on the time elapsed between successive frames and the locations of the object in these successive frames), and the like. Predictor 123, decoder 301, and anomaly detector 401 may be internal to context wrapper 100 as illustrated in FIG. 1 or external to context wrapper 100, as illustrated in FIG. 2.

Predictor 123 and decoder 301 may be trained to predict subsequent images of a scene based on the encoded image data generated by encoder 111 and the encoded motion data encoded by feature extractor/encoder 101. A predicted future state of a scene may be generated by predictor 123 as one or more data points in a latent space (e.g., as in an encoded form), and the one or more data points in the latent space may be decoded into an image by decoder 301. Anomaly detector 401 may subsequently compare the image of the future state of the scene generated by predictor 123 and decoded into an image by decoder 301 against a subsequently captured image of the scene 210. If the predicted image of the scene is substantially similar to the subsequently captured image of the scene 210, anomaly detector 401 can determine that the machine learning models are generating reliable predictions and can continue to use the predictions generated by these machine learning models to trigger various actions (e.g., collision prediction, vehicle control based on collision prediction, etc.). Otherwise, anomaly detector 401 can determine that the machine learning models are operating in an unknown environment and may generate an alert indicating that the models are generating unreliable predictions.

In some aspects, feature extractor/encoder 101 may be trained using unsupervised learning techniques. In such a case, feature extractor/encoder 101 may act as a feature extractor that allows for a reduction in the dimensionality of the model 700 (not shown) that specifies the characteristics of how objects behave in the environment. If feature extractor/encoder 101 is trained using supervised learning techniques, feature extractor/encoder 101 may be coupled with other classification or detection layers that result in the generation of the feature set that represents the characteristics of how objects behave in the environment. Further, in this example, feature extractor/encoder 101 may be trained jointly or separately from encoder 111, predictor 123, and decoder 301 using the contextual information associated with the environment or with other, similar environments (e.g., using transfer learning techniques).

FIGS. 3A through 3C illustrate examples 300A through 300C in which predictions of a future state of objects in an environment are generated based on extracting contextual information about how objects behave in an environment from an input signal.

Signal “A” 310, which may be a single-channel signal or multi-channel signal is input into feature extractor/encoder 101 to encode the signal into a representation (e.g., a feature data set) of characteristics of how an object associated with signal A behaves in an environment. In a medical environment, signal A 310 may be a medical or physiological signal, such as a signal including or derived from medical sensors attached to or otherwise capable of reading vital signs from a patient. Feature extractor 101 may, for example, perform dimensionality reduction on signal A 310 to project signal A 310 into a latent space with lower dimensionality than the dimensionality of signal A 310. A decoder 131 may be trained to reconstruct the encoded signal, representing the characteristics of how the object associated with signal A 310 behaves in an environment, into a representation A′ of the signal. In this example, feature extractor 101 may encode the signal A 310 using various techniques, such as principal component analysis (PCA), bandpass filtering, semantic segmentation of an input signal into an image divided into different segments associated with different objects, or the like. In some aspects, feature extractor/encoder 101 may be a sparse hierarchical encoder, and decoder 131 can be the corresponding decoder, and this sparse hierarchical encoder and decoder can generate the representation of characteristics of how an object associated with signal A behaves in an environment.

Example 300A illustrates an example in which a comparator is used to detect anomalies in signal A 310 and indicate, to a user of context wrapper 100, that context wrapper 100 is being used in an anomalous environment and thus that predictions generated by predictor 123 based on signal A 310 and other input data 320 may be unreliable. Comparator 151 may be configured to monitor for deviations between the representation A′ of the signal and the input signal A 310. Generally, a deviation of signal A′ from signal A 310 that exceeds a defined threshold amount may indicate various problems with the input data generated by the sources of the input data; for example, a deviation may be caused by failure or disconnection of sensors or other components or due to degraded signal quality (e.g., from interference in a system that causes the signal-to-noise ratio to fall below a threshold). When comparator 151 determines that A′ deviates from A 310 by more than a threshold amount, as illustrated in example 300A, an alert may be generated indicating that one or more sources from which signal A is generated have failed or otherwise that the quality of signal A 310 is sufficiently low that the data embodied in signal A, and any resulting predictions made based on signal A 310, may be inaccurate.

In some aspects, as illustrated in example 300B, signal A′ may exclude some components that are present in signal A. For example, where signal A includes noisy components from which no usable data can be extracted, signal A′ many not include such components. Because signal A′ excludes noisy components from the reconstruction of signal A 310, comparator 151 illustrated in FIG. 3A may be omitted, and signal A′ may be provided by a decoder 131 to predictor 123 for use, along with other data 320, in predicting a future state of one or more objects in an environment.

Example 300C illustrates an example in which the provision of signal A 310 into predictor 123 may be controlled based on the representation A′ of signal A 310. In this example, if comparator 151 determines that signal A′ deviates from signal A 310 by more than a threshold amount, comparator 151 may determine that signal A 310 includes unreliable or unusable components. Because signal A 310 includes unreliable or unusable components, switch 601 may, for example, replace signal A with a default or failure signal as an input into predictor 123. By doing so, predictor 123 may generate predictions based on signal A 310 and other data 320 in scenarios in which signal A 310 is considered reliable and may not generate predictions based on signal A 310 and other data 320 in scenarios in which signal A 310 is considered unreliable.

FIG. 4 illustrates an examples 400 in which a physics (or contextual) model 700 is executed within context wrapper 100, according to aspects of the present disclosure. Physics model 700, as discussed, generally specifies various characteristics of how objects behave in an environment in which data is captured. The model may be an analytic model or a computer simulation which operates using classical or quantum physics and may be probabilistic or deterministic. For example, the model 700 may be a Monte Carlo model (a probabilistic simulation model that generates a prediction based on the impact of various random variables), a molecular dynamics model, a particle-in-cell model, coupled rate equations, dynamic models (e.g., defining the properties of solids, liquids, gases, plasmas, granular matter etc.), differential equation-based models, biological or physiological models, genetic models, neural networks, Markov models, control-theory models, digital twin models, or the like.

In example 400, physics model 700 may be executed based on input data 410 optionally filtered using input filter 172 and passed into model 700 via pipeline 171. Generally, the input filter 172 and output filter 176, which are optional features, generally provide data processing, buffering, and conversion capabilities that results in an input of usable data into model 700 and into feature extractor/encoder 101. For example, input filter 172 may transform, normalize, and/or resample the input data, extract metadata from input image(s), and so on. Output filter 176 may, for example stack, concatenate, reshape or resample the output data or images, or restore output data to the original scales or units when the input data was changed (e.g., via a feature normalization procedure) by input filter 172, and so on. As discussed above, feature extractor/encoder 101 generates a feature data set that includes a representation of the various characteristics of how objects behave in an environment and outputs this feature data set to predictor 123 for use, in conjunction with the input data provided to model 700, in predicting a future state of the environment and objects in the environment.

In some aspects, context wrapper 100 and predictor 123 may be used in a surgical environment to predict organ location during a surgical procedure. During a surgical procedure, it may be recognized that the positions of organs within a human body are not fixed, but rather change due to varying factors, such as the position of the patient, heartbeat, breathing, relaxation and constriction of other muscles, edemas, trauma, tumor growth, and the like. Because the positions of organs within the human body are not fixed, performing surgical procedures on a human body based on assumed positions of organs may lead to low accuracy in targeting surgical procedures and may result in tissue damage to healthy tissue.

In this example, the input data may include various samples of medical imagery (e.g., magnetic resonance imaging (MM) scans, computed tomography (CT) scans, etc.), patient pose information, and patient change data (e.g., morbidity, surgical procedure undertaken on the patient, etc.) and other information that may be predictive of a future location of the organs in a patient's body. Model 700 may predict organ movement due to various forces, such as gravity, involuntary muscle movement in the patient's body, surgical changes, the presence or removal of tumors, and the like. The predictions generated by model 700 may be processed by feature extractor/encoder 101 (and optionally decoder 131) to generate a feature data set that represents organ movement within the human body during a surgical procedure. This feature data set may be provided to predictor 123, along with the input data, to generate an image illustrating the predicted locations of the organs in a human body at a future point in time.

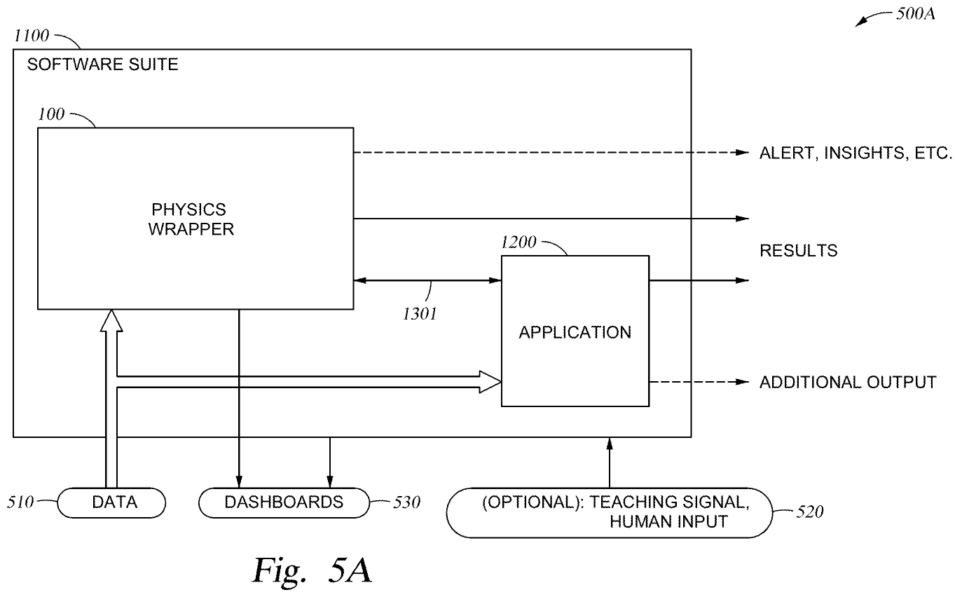

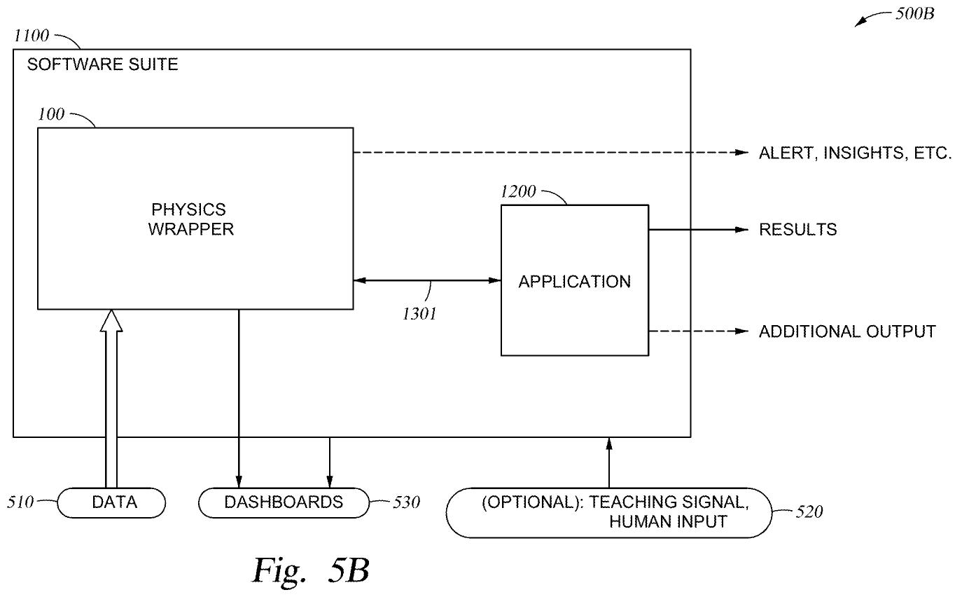

FIGS. 5A and 5B illustrate example pipelines 500A and 500B in which a context-aware machine learning model is deployed, according to aspects of the present disclosure.

In pipeline 500A, context wrapper 100 may be integrated into a software suite 1100. As illustrated, context wrapper 100 provides additional inputs into application 1200 included in software suite 1100 via pipeline 1301 to provide information that may not be available or readily inferred from an original data set. In some aspects, pipeline 1301 may be bidirectional, allowing data to be output from context wrapper 100 as input into application 1200 and allowing data to be output from application 1200 as input into context wrapper 100. When data is output from application 1200 as input into context wrapper 100, the data output from application 122 may be used for various purposes, such as retraining or updating the machine learning models in context wrapper 100 (e.g., a feature extractor/encoder 101 and/or a predictor 123, as discussed above with respect to FIGS. 1 through 4) used to extract the feature data set representing the characteristics of how objects behave in the environment and used to predict a future state of the environment. As illustrated, data 510 may be input directly into both context wrapper 100 and application 1200 for processing. Software suite 1100 may also ingest various signals 520, such as learning signals or human inputs, that can be used in training and controlling use of the machine learning models included in context wrapper 100 and/or application 1200. In some aspects, information about the predicted state of an environment in which software suite 1100 operates, as well as other information generated by software suite 1100 (e.g., via application 1200), may be output to one or more dashboards 530 for visualization and display to a user.

In pipeline 500B, context wrapper 100 may control the data provided as an input into application 1200. Unlike pipeline 500A, in which data 510 is provided to both context wrapper 100 and application 1200, context wrapper 100 may select when to provide data to application 100 for further processing. Such control may, for example, be performed to mitigate overfitting (e.g., when training models used by application 1200) or to prevent application1200 from learning from or otherwise using irrelevant data generated by context wrapper 100 (e.g., due to noise, failures of data capture devices to generate usable data, as discussed above, or the like).

In pipelines 500A and 500B, the models included in context wrapper 100 may be trained separately or jointly with machine learning models included in application 1200. In some aspects, transfer learning can be used to generate the training data sets used to train the machine learning models in context wrapper 100 and application 1200. For example, a context wrapper 100 trained for one application can be used, at least in part, for another application with or without retraining, as appropriate.

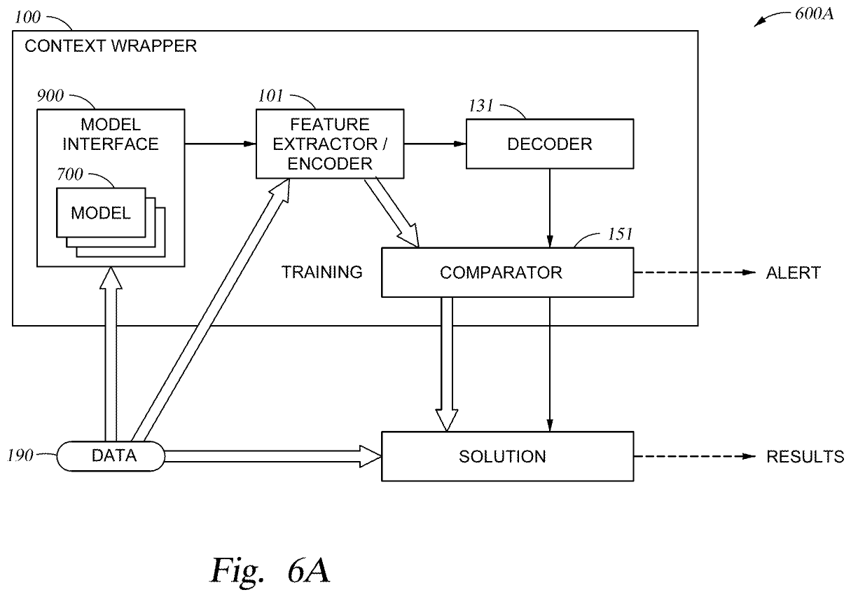

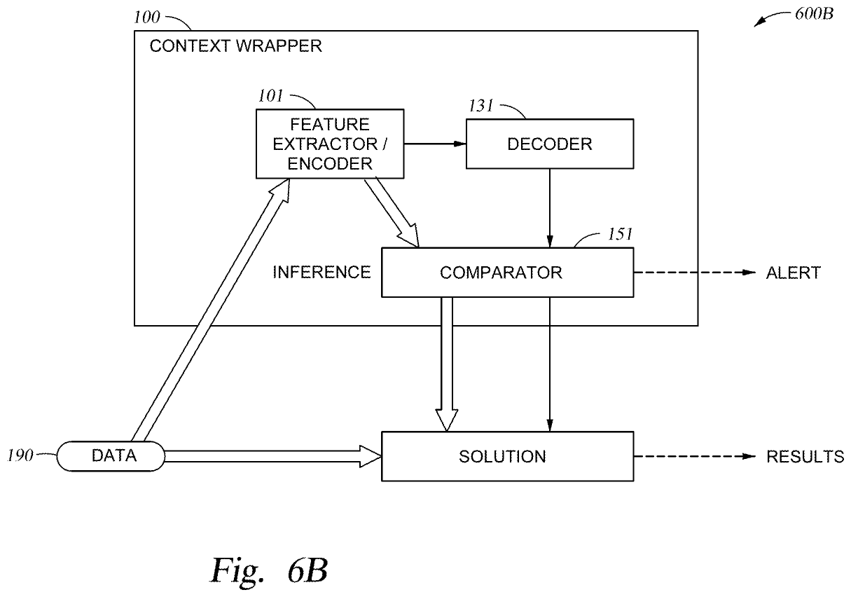

FIGS. 6A and 6B illustrate examples of environments in which context-aware machine learning models are trained and used for inferencing, according to aspects of the present disclosure.

Example 600A illustrates an example of training and testing a context-aware machine learning model. During training and testing, the feature extractor/encoder 101 and decoder 131 may be trained and tested to predict a model output or plurality of model outputs based on a model 700 and input data provided to context wrapper 100. For inference, as illustrated in example 600B, model 700 may be omitted, and the feature extractor/encoder 101 and decoder 131 may predict a model output or plurality of model outputs, given the model input and parameters. Other components, such as predictor 123, can be trained, tested, and used for inference in the same or similar manner. Other components, such as predictor 123, can be trained at the same or subsequent time to the training of the feature extractor / encoder 101 and/or decoder 131.

In some aspects, context wrapper 100 may perform a model parameter search, either adaptive or using a predetermined mesh. This may be used to predict, interpolate, and extrapolate the model behavior at arbitrary parameter values. Boundaries on allowed values of parameters may be provided to context wrapper 100 via various interfaces, such as a command line interface, a graphical user interface, a programmatic interface, an application programming interface (API), or the like. Fixed user parameters, which may include parameters that are not identified by context wrapper 100, can likewise be provided as input to context wrapper 100.

In some aspects, one or more models 700 may be executed multiple times for the same or similar parameter values, for instance using different random value generator seeds. This may be performed, for example, when the behavior or output of the one or more models 700 is not deterministic due to the fundamental properties of the model (e.g. Monte Carlo simulations) or the limited precision of numerical operations. In such cases, components of context wrapper 100, such as feature extractor/encoder 101, decoder 131, predictor 123, and the like, may represent and predict various statistical measures of the output of the one or more models 700, such as the mean, measures of uncertainty of the model output, or a distribution of model outputs, and the like.

### Example Computer Implemented Methods for Training and Using Context-Aware Machine Learning Models to Generate Context-Aware Inferences

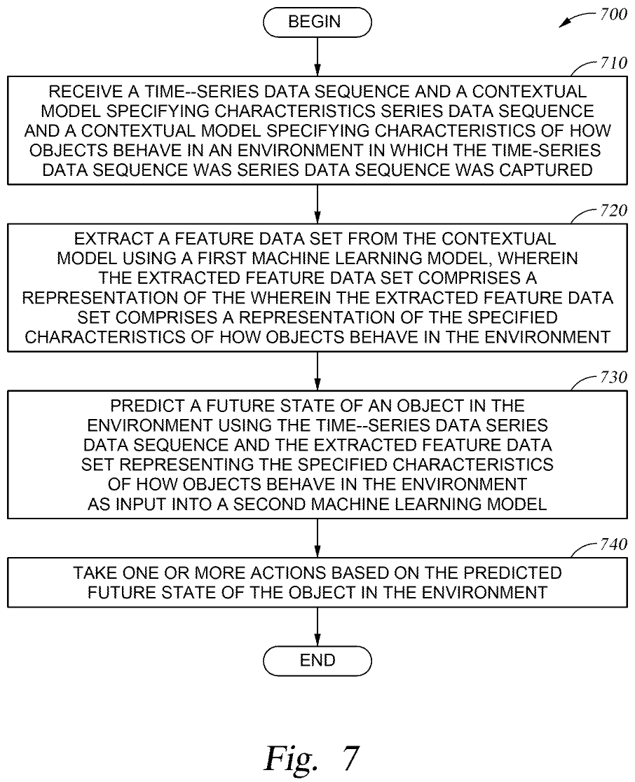

FIG. 7 illustrates example operations 700 for generating inferences using a machine learning model. The operations described herein may be performed by one or more components within a context wrapper, such as feature extractor/encoder 101 and/or predictor 123 illustrated in FIGS. 1 through 6.

As illustrated, operations 700 may begin at block 710, with receiving a time-series data sequence and a contextual model specifying characteristics of how objects behave in an environment in which the time-series data sequence was captured.

At block 720, operations 700 proceed with extracting a feature data set from the contextual model using a first machine learning model. Generally, the feature data set may include a representation of the specified characteristics of how objects behave in the environment. The feature data set may be generated with reduced dimensionality relative to the contextual model.

In some aspects, the first machine learning model may be an encoder-decoder model. The encoder may be trained to extract the feature data set as one or more points in a feature space (or latent space). The decoder, meanwhile, may be trained to construct an approximation of a scene from the feature data set.

At block 730, operations 700 proceed with predicting a future state of an object in the environment using the time-series data sequence and the extracted feature data set representing the specified characteristics of how objects behave in the environment as input into a second machine learning model.

In some aspects, the second machine learning model may be an encoder-decoder model. The encoder may be trained to encode an input into a feature space. A predictor may be trained to predict the future state of the time-series data sequence based on the encoded input and the feature data set. Finally, the decoder may be trained to construct an approximation of a scene from the predicted future state of the time-series data sequence.

At block 740, operations 700 proceed with taking one or more actions based on the predicted future state of the object in the environment.

In some aspects, taking the one or more actions includes calculating a prediction error between an actual future state of the environment and the predicted future state of the environment. Based on determining that the calculated prediction error exceeds a threshold prediction error, an alert may be generated to indicate that the time-series data sequence comprises an unknown data set for which predictions will be unreliable. As discussed, such an indication may indicate that the data set includes data from a new scenario in a different environment previously unseen in the training data set, degraded or unrecognizable data from one or more sensors providing data as an input into the machine learning models, or the like.

In some aspects, to calculate the prediction error, a prediction error heat map may be generated. The prediction error heat map includes a plurality of segments, with each segment being associated with a difference between a respective segment of the plurality of segments in an image representing the predicted future state and an image representing the actual future state. An error score is subsequently calculated based on a value of each segment of the plurality of segments in the prediction error heat map.

In some aspects, the predicted future state of the object in the environment may be a predicted operational state of a machine. A calculated prediction error in such a case may in clue a difference between a predicted operational state of the machine and an actual operational state of the machine at a future point in time. Generally, where the predicted and actual operational state of the machine at this future point in time are similar, it may be determined that the machine is operating normally. Otherwise, it may be determined that the machine is potentially operating abnormally and thus that corrective action should be taken.

In some aspects, the predicted future state of the object in the environment may be a predicted set of vital signs for a patient (e.g., a patient on which a surgical procedure is being performed, a patient being treated for a medical condition, etc.). A calculated prediction error may be a difference between the predicted set of vital signs for the patient and the actual vital signs for the patient at a future point in time. Generally, where the predicted and actual vital signs are similar, no further interventions need be taken. However, where the predicted and actual vital signs diverge, it may be determined that some medical intervention should be performed for the patient.

In some aspects, the contextual model includes a physics model defining rules for how objects move in relation to other objects in the environment. The environment may be a physical environment or a virtual environment. When the environment is a physical environment, the predicted future state of the object may be a location and orientation of the object in the physical environment. When the environment is a virtual environment, the predicted future state of the object may include properties of the object in the virtual environment.

In some aspects, the time-series data includes historical disease progression data for a patient. The contextual model may specify how a disease progresses over time. The predicted future state of the object may be a predicted stage of a disease in a future point in time. In such a case, a prediction error may indicate a pause in disease progression or an acceleration in disease progression relative to a baseline. A prediction error in excess of some threshold may trigger a change in a treatment plan for the patient.

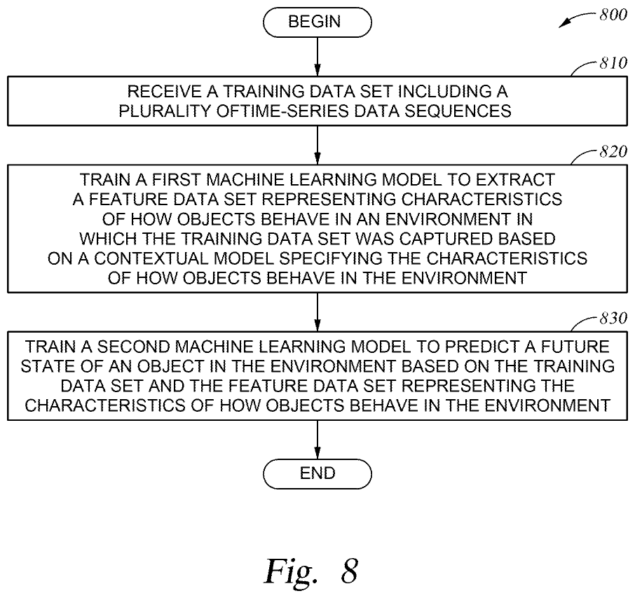

FIG. 8 illustrates example operations that may be performed by a computing system to train machine learning models, such as feature extractor/encoder 101 and/or predictor 123 illustrated in FIGS. 1 through 6, which generate context-aware inferences.

As illustrated, operations 800 begin at block 810, with receiving a training data set including a plurality of time-series data sequences. In some aspects, each time-series data sequence may include a plurality of images in a scene captured sequentially from a start time to an end time. In some aspects, the time-series data sequence may include a set of data points captured over time, such as patient vital signs captured over time, machine operational statistics or data captured over time, or other data relevant to an environment in which the machine learning models are used to predict the future state of that environment.

At block 820, operations 800 proceed with training a first machine learning model to extract a feature data set based on a contextual model specifying characteristics of how objects behave in the environment. Generally, the feature data set may be a feature data set with a lower dimensionality than a dimensionality associated with the contextual model. The feature data set may include, for example, a set of features and feature coefficients as a function of time. In some aspects, the first machine learning model may include an encoder-decoder model including an encoder trained to extract the feature data set as one or more points in a feature space and a decoder trained to construct an approximation of a scene from the feature data set.

At block 830, operations 800 proceed with training a second machine learning model to predict a future state of an object in the environment based on the training data set and the feature data set representing the characteristics of how objects behave in the environment. In some aspects, the second machine learning model may include an encoder-decoder model including an encoder trained to encode an input into a feature space, a predictor trained to predict the future state of the time-series data sequence based on the encoded input and the feature data set, and a decoder trained to construct an approximation of a scene from the predicted future state of the time-series data sequence. In some aspects, in order to train the second machine learning model, a difference between the predicted future state of the time-series data sequence and the time-series data sequence in the training data set may be minimized.

The first machine learning model and the second machine learning model may be trained using various techniques. In some aspects, the first and second machine learning models may be trained using unsupervised learning techniques. In some aspects, the first and second machine learning models may be trained using self-supervised learning techniques.

### Example System for Training and Using Context-Aware Machine Learning Models to Generate Context-Aware Inferences

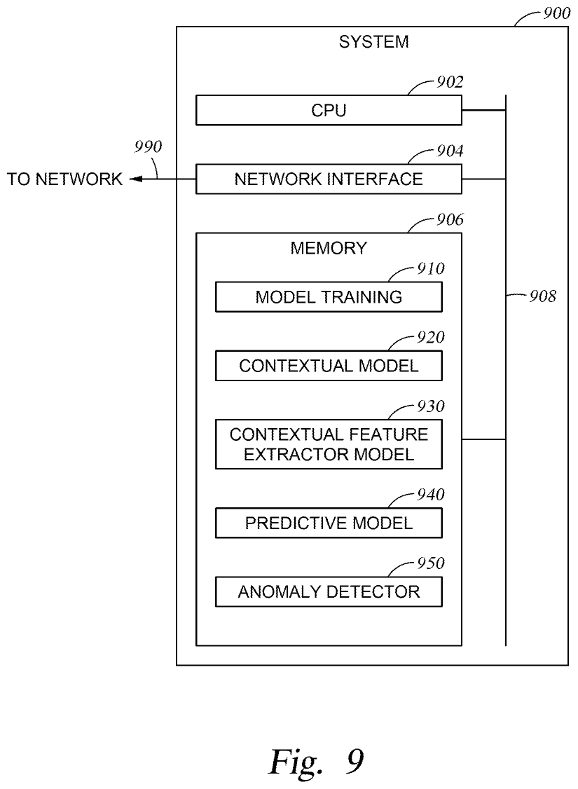

FIG. 9 illustrates an example system 900 configured to perform the methods described herein, including, for example, operations 700 of FIG. 7 and/or operations 800 of FIG. 8. In some embodiments, system 900 may act as a computing system on which a context wrapper including a feature extractor/encoder model (e.g., feature extractor/encoder 101 illustrated in FIG. 1) and a prediction model (e.g., predictor 123 illustrated in FIG. 1) are trained and used to generate context-aware inferences.

As shown, system 900 includes a central processing unit (CPU) 902, a network interface 904 through which system 900 is connected to network 990 (which may be a local network, an intranet, the interne, or any other group of computing devices communicatively connected to each other), and a memory 906, connected via an interconnect 908.

CPU 902 may retrieve and execute programming instructions stored in the memory 906. Similarly, the CPU 902 may retrieve and store application data residing in the memory 906. The interconnect 908 transmits programming instructions and application data, among the CPU 902, network interface 904, and memory 906.

CPU 902 is included to be representative of a single CPU, multiple CPUs, a single CPU having multiple processing cores, and the like.

Memory 906 is representative of a volatile memory, such as a random access memory, or a nonvolatile memory, such as nonvolatile random access memory, phase change random access memory, or the like. As shown, memory 906 includes a model trainer 910, contextual model 920, contextual feature extractor model 930, predictive model 940, and anomaly detector 950.

Model trainer 910 generally is configured to train the contextual feature extractor model 930 and predictive model 940 based on a training data set of time-series data samples and a contextual model 920 that defines the characteristics of object behavior within an environment in which inferences are performed. The contextual feature extractor model 930 and predictive model 940 may be trained using unsupervised learning techniques or semi-supervised learning techniques.

Contextual feature extractor model 930 may correspond to feature extractor/encoder 101 illustrated in FIGS. 1 through 6 and may be trained to generate a feature data set that comprises a reduced-dimensionality representation of the specified characteristics of how objects behave in the environment. The contextual feature extractor model 930 may be, in some aspects, an encoder-decoder model trained to encode an input into a feature space and a decoder trained to construct an approximation of a scene from the feature data set.

Predictive model 940 may correspond to predictor 123 illustrated in FIGS. 1 through 6. Predictive model 940 is generally trained to predict a future state of objects in an environment based on the feature data set extracted from a contextual model by contextual feature extractor model 930 and an input data set of time-series data from the environment. Predictive model 940 may be structured, in some aspects, as an encoder-decoder model include an encoder trained to encode an input into a feature space, a predictor trained to predict the future state of the time-series data sequence based on the encoded input and the feature data set, and a decoder trained to construct an approximation of a scene from the predicted future state of the time-series data sequence.

### Example Clauses

Implementation details of various aspects of the present disclosure are described in the following numbered clauses.

Clause 1: A computer-implemented method for generating inferences using a machine learning model, comprising: receiving a time-series data sequence and a contextual model specifying characteristics of how objects behave in an environment in which the time-series data sequence was captured; extracting a feature data set from the contextual model using a first machine learning model, wherein the extracted feature data set comprises a representation of the specified characteristics of how objects behave in the environment; predicting a future state of an object in the environment using the time-series data sequence and the extracted feature data set representing the specified characteristics of how objects behave in the environment as input into a second machine learning model; and taking one or more actions based on the predicted future state of the object in the environment.

Clause 2: The method of Clause 1, wherein the first machine learning model comprises an encoder-decoder model including an encoder trained to extract the feature data set as one or more points in a feature space and a decoder trained to construct an approximation of a scene from the feature data set.

Clause 3: The method of any one of Clauses 1 or 2, wherein the second machine learning model comprises an encoder-decoder model including an encoder trained to encode an input into a feature space, a predictor trained to predict the future state of the time-series data sequence based on the encoded input and the feature data set, and a decoder trained to construct an approximation of a scene from the predicted future state of the time-series data sequence.

Clause 4: The method of any one of Clauses 1 through 3, wherein taking the one or more actions comprises: calculating a prediction error between an actual future state of the environment and the predicted future state of the environment; and based on determining that the calculated prediction error exceeds a threshold prediction error, generating an alert indicating the time-series data sequence comprises an unknown data set for which predictions will be unreliable.

Clause 5: The method of Clause 4, wherein calculating the prediction error comprises: generating a prediction error heat map including a plurality of segments, each segment being associated with a difference between a respective segment of the plurality of segments in an image representing the predicted future state and an image representing the actual future state; and calculating an error score based on a value of each segment of the plurality of segments in the prediction error heat map.

Clause 6: The method of any one of Clauses 4 or 5, wherein the predicted future state comprises a predicted operational state of a machine and the calculated prediction error comprises a difference between the predicted operational state of the machine and an actual operational state of the machine at a future point in time.

Clause 7: The method of any one of Clauses 4 through 6, wherein the predicted future state comprises a predicted set of vital signs for a patient and the calculated prediction error comprises a difference between the predicted set of vital signs for the patient and actual vital signs for the patient at a future point in time.

Clause 8: The method of any one of Clauses 1 through 7, wherein the first machine learning model is trained to generate the feature data set with reduced dimensionality relative to the contextual model.

Clause 9: The method of any one of Clauses 1 through 8, wherein the contextual model comprises a physics model defining rules for how objects move in relation to other objects in the environment.

Clause 10: The method of any one of Clauses 1 through 9, wherein the time-series data sequence comprises historical disease progression data for a patient, the contextual model specifies how a disease progresses over time, and the predicted future state of the object comprises a predicted stage of a disease at a future point in time.

Clause 11: The method of any one of Clauses 1 through 10, wherein the environment comprises a physical environment, and the predicted future state of the object comprises a location and orientation of the object in the physical environment.

Clause 12: The method of any one of Clauses 1 through 11, wherein the environment comprises a virtual environment, and the predicted future state of the object comprises properties of the object in the virtual environment.

Clause 13: A computer-implemented method for training machine learning models, comprising: receiving a training data set including a plurality of time-series data sequences; training a first machine learning model to extract a feature data set representing characteristics of how objects behave in an environment in which the training data set was captured based on a contextual model specifying the characteristics of how objects behave the environment; and training a second machine learning model to predict a future state of an object in the environment based on the training data set and the feature data set representing the characteristics of how objects behave in the environment.

Clause 14: The method of Clause 13, wherein the first machine learning model comprises an encoder-decoder model including an encoder trained to extract the feature data set as one or more points in a feature space and a decoder trained to construct an approximation of a scene from the feature data set.

Clause 15: The method of Clause 14, wherein the feature data set comprises a set of features and feature coefficients as a function of time.

Clause 16: The method of any one of Clauses 13 through 15, wherein the second machine learning model comprises an encoder-decoder model including an encoder trained to encode an input into a feature space, a predictor trained to predict the future state of a time-series data sequence based on the encoded input and the feature data set, and a decoder trained to construct an approximation of a scene from the predicted future state of the time-series data sequence.

Clause 17: The method of any one of Clauses 13 through 16, wherein training the second machine learning model comprises training the second machine learning model to minimize a difference between the predicted future state of a time-series data sequence and the time-series data sequences in the training data set.

Clause 18: The method of any one of Clauses 13 through 17, wherein the first machine learning model and the second machine learning model are trained using self-supervised learning techniques.

Clause 19: The method of any one of Clauses 13 through 18, wherein each time-series data sequence comprises a plurality of images of a scene captured sequentially from a start time to an end time.

Clause 20: A method for providing additional information to a system, the system comprising a physics model or plurality of physics models, a model interface, a feature extractor/encoder, and plurality of pipelines and interfaces, such that the physics model or plurality of physics models provide information to the system or device which is not contained, or contained but not explicitly specified, in original data or features used to train and/or operate the system, whereby provision of the additional information improves performance indicators of the system.

Clause 21: The method of Clause 20, wherein the additional information restricts the set of features used or learned by the system.

Clause 22: The method of any one of Clauses 20 or 21, wherein the additional information augments the input data provided to the system.

Clause 23: The method of any one of Clauses 20 through 22, wherein a decoder is used in conjunction with the feature extractor/encoder to transform and/or reduce the dimensionality of some of all of the data and/or to make pertinent information contained in the data more accessible.

Clause 24: The method of any one of Clauses 20 through 23, further comprising one or more anomaly detectors.

Clause 25: The method of Clause 24, wherein the one or more anomaly detectors is used autonomously or with a human in the loop to provide a signal or a plurality of signals to indicate that the system is operating (recently, currently, and/or in the future) using unexpected or abnormal inputs or that the operating is operating in an unexpected or abnormal state.

Clause 26: The method of any one of Clauses 20 through 25, further comprising: providing additional information to the system, wherein the physical model or plurality of physical models are implemented and/or executed independently and output results via an interface.

Clause 27: The method of Clause 26, wherein the interface is configured to transform, filter, subsample, buffer, compress, or otherwise modify the data produced by the physics model of the plurality of physics models.

Clause 28: The method of any one of Clauses 20 through 27, further comprising a predictor trained based on the data and the physics model to predict future inputs and/or future state of the physics model.

Clause 29: The method of Clause 28, wherein the prediction is based, at least in part, on the future state of the physics model.

Clause 30: The method of any one of Clauses 20 through 29, wherein at least one interface of the plurality of interfaces comprises an interface to a sensor or plurality of sensors, the feature extractor/encoder, and the plurality of pipelines, such that the sensor or plurality of sensors provide the information to the system.

Clause 31: The method of any one of Clauses 20 through 30, wherein the interface comprises an interface to a game engine or virtual reality system of simulation, and wherein the game engine or virtual reality simulation to the system.

Clause 32: The method of any one of Clauses 20 through 31, wherein the system implements one or more artificial neural networks trained for one or more of medical system or device operations (computer-aided diagnostics, triage, or treatment), autonomous vehicle control, robot control, security, surveillance, search and rescue, and/or anomaly detection.

Clause 33: The method of any one of Clauses 20 through 32, further comprising one or more of a safety device or system, a financial system, an industrial system, or interne of things (IoT) system.

Clause 34: The method of any one of Clauses 20 through 33, wherein the information is further used to ensure safe operation of the system or trigger safety procedures in the system (e.g., alarming, alerting, warning, backing up, entering into a safe mode, shutting down, landing, surfacing, slowing down, or adjusting operation modes or parameters of the system).

Clause 35: The method of any one of Clauses 20 through 34, wherein the information prevents or corrects an erroneous or suboptimal action or output of the system and provides a teaching signal such that the erroneous or suboptimal action or output is less likely to occur in the future.

Clause 36: The method of any one of Clauses 20 through 34, wherein the information is used by multiple systems via federated learning of one or more models.

Clause 37: A processing system, comprising: a memory having executable instructions stored thereon; and a processor configured to execute the executable instructions to cause the processing system to perform the operations of any one of Clauses 1 through 36.

Clause 38: A processing system, comprising: means for performing the operations of any one of Clauses 1 through 36.

Clause 39: A computer-readable medium having executable instructions stored thereon which, when executed by a processor, causes the processor to perform the operations of any one of clauses 1 through 36.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A computer-implemented method for generating inferences using a machine learning model, comprising:
receiving a time-series data sequence and a contextual model specifying characteristics of how objects behave in an environment in which the time-series data sequence was captured;
extracting a feature data set from the contextual model using a first machine learning model, wherein the extracted feature data set comprises a representation of the specified characteristics of how objects behave in the environment;
predicting a future state of an object in the environment using the time-series data sequence and the extracted feature data set representing the specified characteristics of how objects behave in the environment as input into a second machine learning model; and
taking one or more actions based on the predicted future state of the object in the environment.

2. The method of claim 1, wherein the first machine learning model comprises an encoder-decoder model including an encoder trained to extract the feature data set as one or more points in a feature space and a decoder trained to construct an approximation of a scene from the feature data set.

3. The method of claim 1, wherein the second machine learning model comprises an encoder-decoder model including an encoder trained to encode an input into a feature space, a predictor trained to predict the future state of the time-series data sequence based on the encoded input and the feature data set, and a decoder trained to construct an approximation of a scene from the predicted future state of the time-series data sequence.

4. The method of claim 1, wherein taking the one or more actions comprises:
calculating a prediction error between an actual future state of the environment and the predicted future state of the environment; and
based on determining that the calculated prediction error exceeds a threshold prediction error, generating an alert indicating the time-series data sequence comprises an unknown data set for which predictions will be unreliable.

5. The method of claim 4, wherein calculating the prediction error comprises:
generating a prediction error heat map including a plurality of segments, each segment being associated with a difference between a respective segment of the plurality of segments in an image representing the predicted future state and an image representing the actual future state; and
calculating an error score based on a value of each segment of the plurality of segments in the prediction error heat map.

6. The method of claim 4, wherein the predicted future state comprises a predicted operational state of a machine and the calculated prediction error comprises a difference between the predicted operational state of the machine and an actual operational state of the machine at a future point in time.

7. The method of claim 4, wherein the predicted future state comprises a predicted set of vital signs for a patient and the calculated prediction error comprises a difference between the predicted set of vital signs for the patient and actual vital signs for the patient at a future point in time.

8. The method of claim 1, wherein the first machine learning model is trained to generate the feature data set with reduced dimensionality relative to the contextual model.

9. The method of claim 1, wherein the contextual model comprises a physics model defining rules for how objects move in relation to other objects in the environment.

10. The method of claim 1, wherein the time-series data sequence comprises historical disease progression data for a patient, the contextual model specifies how a disease progresses over time, and the predicted future state of the object comprises a predicted stage of a disease at a future point in time.

11. The method of claim 1, wherein the environment comprises a physical environment, and the predicted future state of the object comprises a location and orientation of the object in the physical environment.

12. The method of claim 1, wherein the environment comprises a virtual environment, and the predicted future state of the object comprises properties of the object in the virtual environment.

13. A computer-implemented method for training machine learning models, comprising:
receiving a training data set including a plurality of time-series data sequences;
training a first machine learning model to extract a feature data set representing characteristics of how objects behave in an environment in which the training data set was captured based on a contextual model specifying the characteristics of how objects behave the environment; and
training a second machine learning model to predict a future state of an object in the environment based on the training data set and the feature data set representing the characteristics of how objects behave in the environment.

14. The method of claim 13, wherein the first machine learning model comprises an encoder-decoder model including an encoder trained to extract the feature data set as one or more points in a feature space and a decoder trained to construct an approximation of a scene from the feature data set.

15. The method of claim 14, wherein the feature data set comprises a set of features and feature coefficients as a function of time.

16. The method of claim 13, wherein the second machine learning model comprises an encoder-decoder model including an encoder trained to encode an input into a feature space, a predictor trained to predict the future state of a time-series data sequence based on the encoded input and the feature data set, and a decoder trained to construct an approximation of a scene from the predicted future state of the time-series data sequence.

17. The method of claim 13, wherein training the second machine learning model comprises training the second machine learning model to minimize a difference between the predicted future state of a time-series data sequence and the time-series data sequences in the training data set.

18. The method of claim 13, wherein the first machine learning model and the second machine learning model are trained using self-supervised learning techniques.

19. The method of claim 13, wherein each time-series data sequence comprises a plurality of images of a scene captured sequentially from a start time to an end time.

20. A processing system, comprising:
a memory having executable instructions stored thereon; and
a processor configured to execute the executable instructions to cause the processing system to:
receive a time-series data sequence and a contextual model specifying characteristics of how objects behave in an environment in which the time-series data sequence was captured;
extract a feature data set from the contextual model using a first machine learning model, wherein the extracted feature data set comprises a representation of the specified characteristics of how objects behave in the environment;
predict a future state of an object in the environment using the time-series data sequence and the extracted feature data set representing the specified characteristics of how objects behave in the environment as input into a second machine learning model; and
take one or more actions based on the predicted future state of the object in the environment.
